(19) United States
(12) Patent Application Publication
(10) Pub. No.: US 2020/0168327 A1
(43) Pub. Date: May 28, 2020

(54) CUSTOMIZABLE COMMUNICATION PLATFORM WITH JOURNAL LOG

(71) Applicant: CAREMINDR Corporation, Los Gatos, CA (US)

(72) Inventors: Harry Raymond Soza, San Jose, CA (US); Irina Yermilov, Los Angeles, CA (US)

(21) Appl. No.: 16/696,673

(22) Filed: Nov. 26, 2019

Related U.S. Application Data
(60) Provisional application No. 62/771,919, filed on Nov. 27, 2018.

(51) Int. Cl. G16H 40/63; G16H 40/20

(52) U.S. Cl. CPC G16H 40/63; G16H 40/20

(57) ABSTRACT

One example method of patient biometric data analysis includes one or more of, requesting via a cloud server at least one patient response to at least one message sent to a wireless device including at least one query of at least one health related issue, receiving at least one patient response to the at least one query, determining at least one objective data historical trend of the at least one response, determining at least one subjective data historical trend of the at least one response, providing via the cloud server at least one urgent tagged historical trend based on at least one of the at least one objective data historical trend and the at least one subjective data historical trend and displaying the at least one urgent tagged historical trend.

### TECHNICAL FIELD OF THE APPLICATION

This application relates to a customizable communication platform and more particularly to providing customized healthcare communication to a user device by integrating various personal records with an ongoing communication regiment, wherein the communication includes tags pertaining to urgent healthcare issues.

### BACKGROUND OF THE APPLICATION

Conventionally, the approach to providing users with ongoing communications regarding a plan or other repetitive course of action may leave the majority of the work to the user. The smartphone and other personal computing devices are everywhere and are not being properly utilized when offering users with options for maintaining a course of treatment or a set of goals. The lack of action taken by the professional service provider and/or the user can lead to personal health problems and lost revenue for providers, insurers, etc., as well as the users.

### SUMMARY OF THE APPLICATION

Example embodiments of the present application provide at least a first method that includes at least one of requesting via a cloud server at least one patient response to at least one message sent to a wireless device including at least one query of at least one health related issue, receiving at least one patient response to the at least one query, determining at least one objective data historical trend of the at least one response, determining at least one subjective data historical trend of the at least one response, providing via the cloud server at least one urgent tagged historical trend based on at least one of the at least one objective data historical trend and the at least one subjective data historical trend and displaying the at least one urgent tagged historical trend.

A second example non-transitory computer readable medium of the present application comprises instructions that, when read by a processor, cause the processor to perform at least one of, requesting via a cloud server at least one patient response to at least one message sent to a wireless device including at least one query of at least one health related issue, receiving at least one patient response to the at least one query, determining at least one objective data historical trend of the at least one response, determining at least one subjective data historical trend of the at least one response, providing via the cloud server at least one urgent tagged historical trend based on at least one of the at least one objective data historical trend and the at least one subjective data historical trend and displaying the at least one urgent tagged historical trend.

A third example embodiment of the present application provides a system, comprising, at least one cloud based processor, and at least one memory electrically coupled to the at least one processor and storing an application, wherein the processor performs operations to perform at least one of, request via a cloud server at least one patient response to at least one message sent to a wireless device including at least one query of at least one health related issue, receive at least one patient response to the at least one query, determine at least one objective data historical trend of the at least one response, determine at least one subjective data historical trend of the at least one response, provide via the cloud server at least one urgent tagged historical trend based on at least one of the at least one objective data historical trend and the at least one subjective data historical trend and display the at least one urgent tagged historical trend.

### BRIEF DESCRIPTION OF THE DRAWINGS

FIG. 1 illustrates an example of the integrated application platform according to example embodiments.

FIG. 2 illustrates a network configuration of the third party participants of the integrated application according to example embodiments.

FIG. 3 illustrates a user smartphone interface of an example treatment plan according to example embodiments.

FIG. 4A illustrates an example setup configuration for a new course of treatment according to example embodiments.

FIG. 4B illustrates an example database entry for the new course of treatment according to example embodiments.

FIG. 4C illustrates a flow diagram configuration for the new course of treatment according to example embodiments.

FIG. 4D illustrates an example list of messages for the ongoing course of treatment according to example embodiments.

FIG. 4E illustrates an example setup configuration for various courses of treatment according to example embodiments.

FIG. 4F illustrates an example set of details of an ongoing course of treatment according to example embodiments.

FIG. 4G illustrates an example network configuration of the various third parties involved in the application operation and compliance according to example embodiments.

FIG. 5 illustrates a logic module configured to process the input and output parameters of the application according to example embodiments.

FIG. 6 illustrates an example network entity device configured to store instructions, software, and corresponding hardware for executing the same, according to example embodiments of the present application.

FIG. 7 illustrates a data file defining tags and levels of urgency, according to example embodiments of the present application.

FIG. 8 illustrates a patient reply, according to example embodiments of the present application.

FIG. 9 illustrates an electronic report to a healthcare provider with alert tags, according to example embodiments of the present application.

FIG. 10 illustrates system architecture, according to example embodiments of the present application.

FIG. 11 illustrates a first method, according to example embodiments of the present application.

FIG. 12 illustrates a second method, according to example embodiments of the present application.

FIG. 13 depicts a screenshot showing the journey log app, according to example embodiments of the present application.

FIG. 14 depicts a screenshot showing the journey log data output, according to example embodiments of the present application.

FIG. 15 depicts a screenshot showing the journey log app and associated data output according to example embodiments of the present application.

FIG. 16 depicts a screenshot showing the journey log medical professional data output, according to example embodiments of the present application.

FIG. 17 depicts a first journey log method, according to example embodiments of the present application.

FIG. 18 depicts a second journey log method, according to example embodiments of the present application.

FIG. 19 depicts a third journey log method, according to example embodiments of the present application.

FIG. 20 depicts a fourth journey log method, according to example embodiments of the present application.

### DETAILED DESCRIPTION OF THE APPLICATION

It will be readily understood that the components of the present application, as generally described and illustrated in the figures herein, may be arranged and designed in a wide variety of different configurations. Thus, the following detailed description of the embodiments of a method, apparatus, and system, as represented in the attached figures, is not intended to limit the scope of the application as claimed, but is merely representative of selected embodiments of the application.

The features, structures, or characteristics of the application described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present application. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

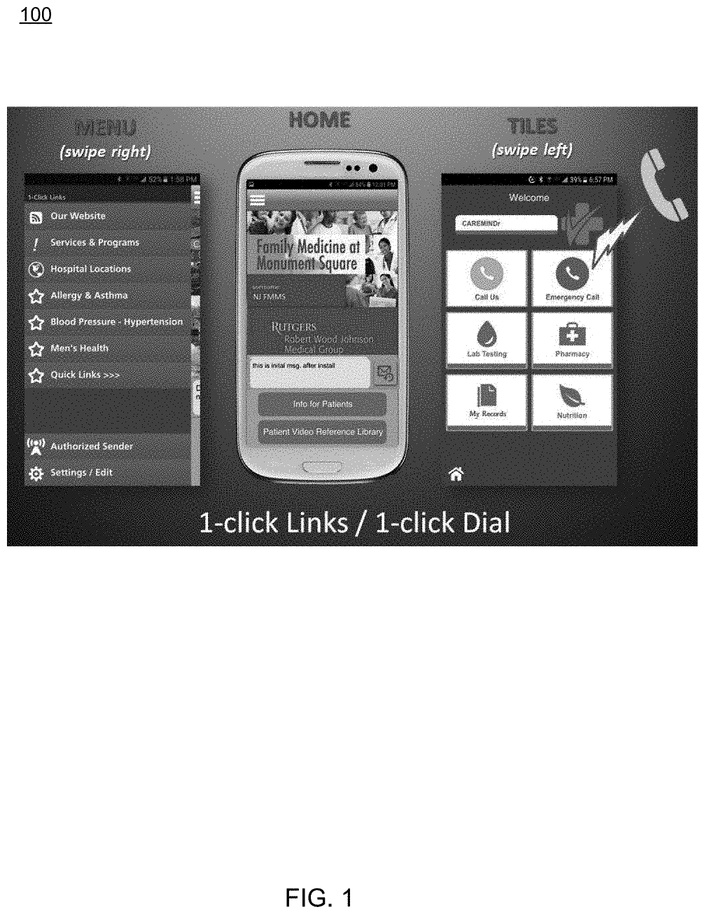

FIG. 1 illustrates an example of the integrated application platform according to example embodiments. Referring to FIG. 1, the configuration 100 includes a menu user interface, a home user interface and a set of option tiles for accessing third party resources, such as test results, emergency concerns, pharmacy information, etc.

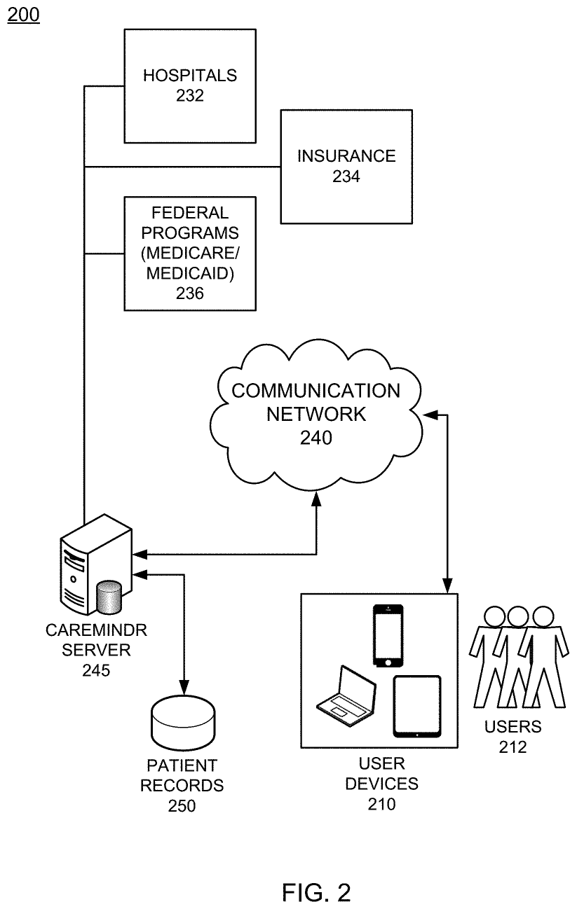

FIG. 2 illustrates a network configuration of the third party participants of the integrated application according to example embodiments. Referring to FIG. 2, the network includes a central server 245 with patient records 250. The information needed to provide treatment plans and other integrated services may require access to hospital and other provider services 232, insurance company information 234, drug providers, federal program administrators 236, etc. The information may be incorporated into any treatment plan or other integrated service model accessed by a user device 210 operated by a user 212. The servers and third party modules may operate on-site or in a cloud network managed by the providers.

Examples of treatment plans and other objectives may include a care management service for assessment of patient medical needs. The system and application may ensure timely receipt of all recommended treatment actions, drugs, third party services and over a designated period of time. Also, referrals to other providers and additional services may provide emergency visits, discharge instructions, nursing facility operations, and home health care functions. In operation, the procedure may begin with the medical treatment provider creating a treatment plan or ‘journey’ for each patient. Each journey is generally for a single chronic condition or objective. One patient may have multiple journeys integrated into a single application. Also, the journeys may originate from various different providers and service entities. The journey will provide the healthcare provider with biometric, objective and subjective data to enable evidence-based medical decisions. As an example, the biometric data may be glucometer data collected from a blue tooth enabled device and made available to the physician, objective data such as whether the patient visited an emergency room or hospital and subjective data such as how the patient is feeling.

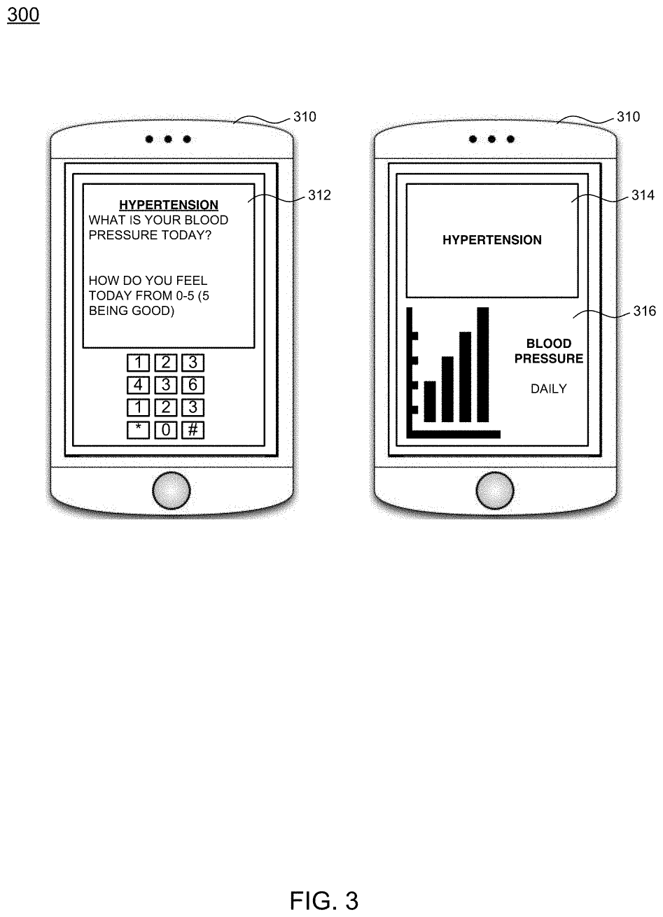

FIG. 3 illustrates a user smartphone interface of an example treatment plan according to example embodiments. Referring to FIG. 3, the journey for “hypertension” may have been created or modified by a patient doctor and may include an interface 300 with a smartphone device 310 and a screen option configuration providing questions 312, information about the treatment, reminders and other functions. The example in FIG. 3 provides for a set of questions 312 and a journey topic 314 along with a graph of blood pressure records 316 as measured over time from various interactions.

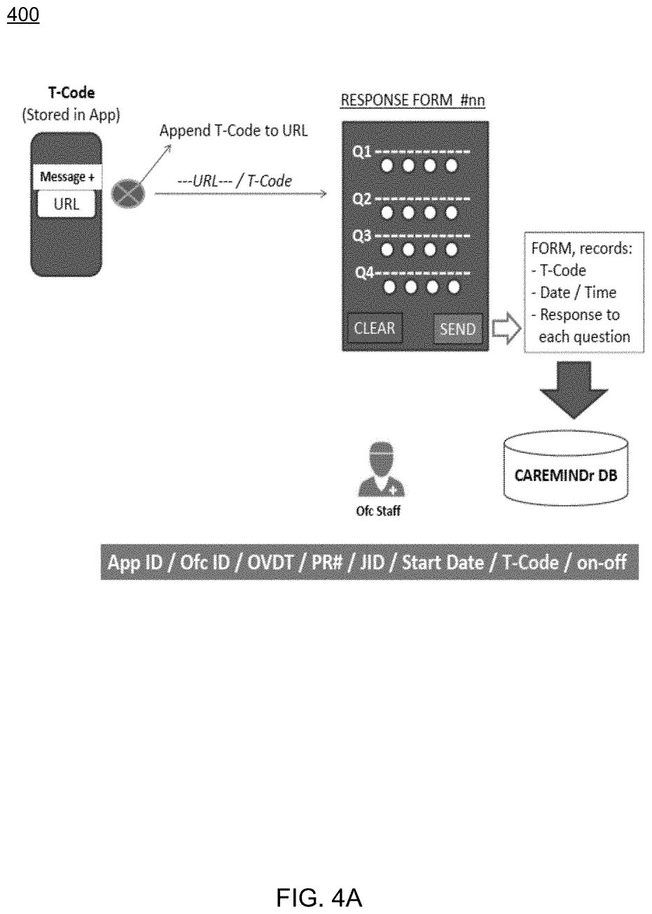

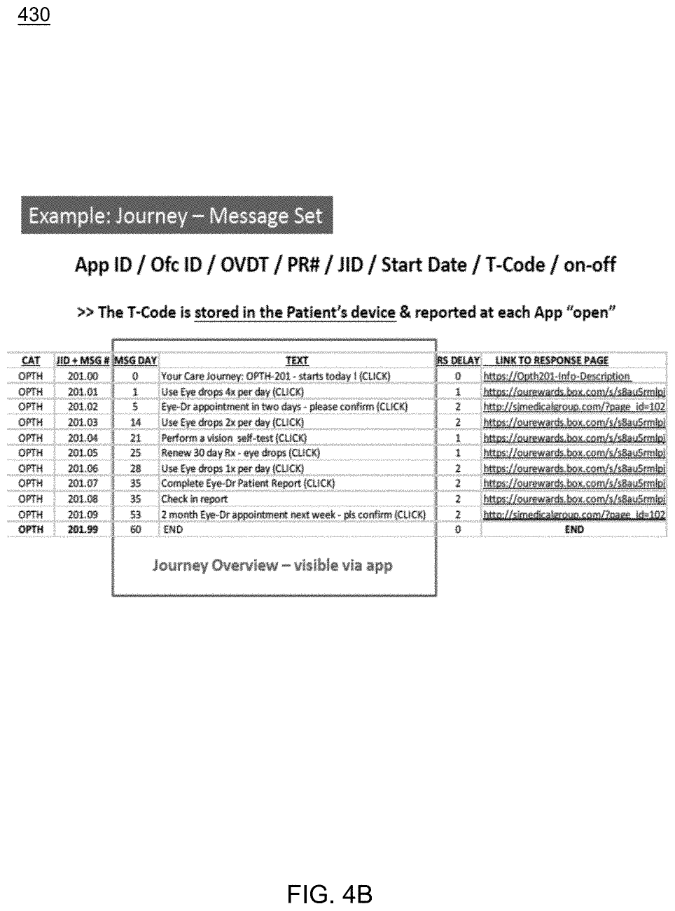

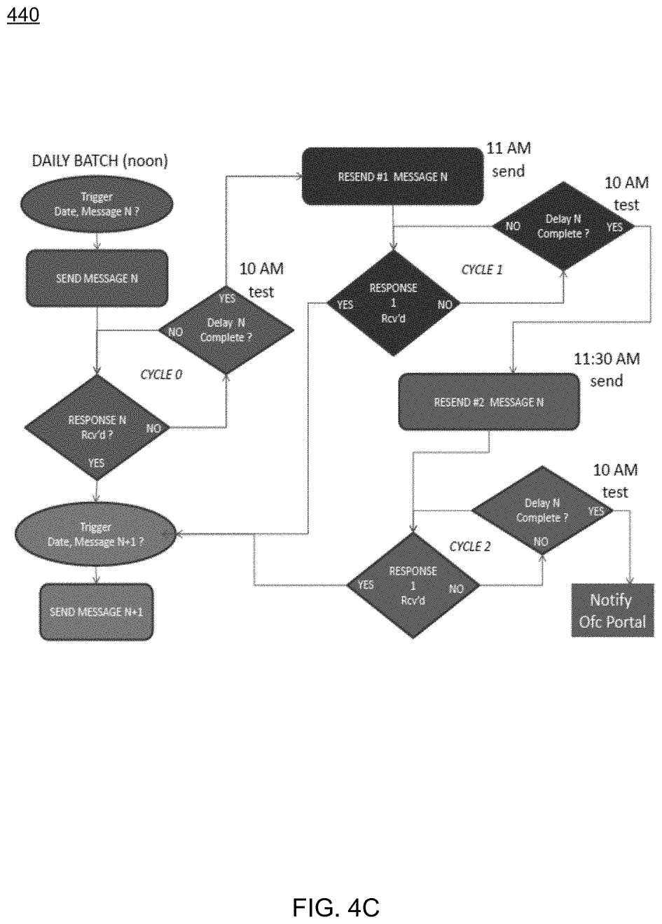

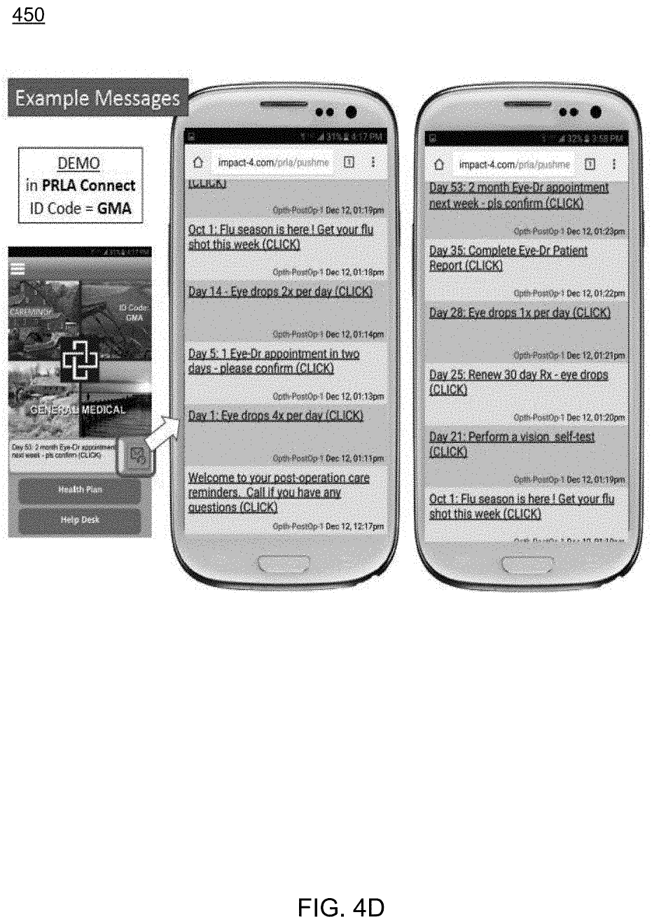

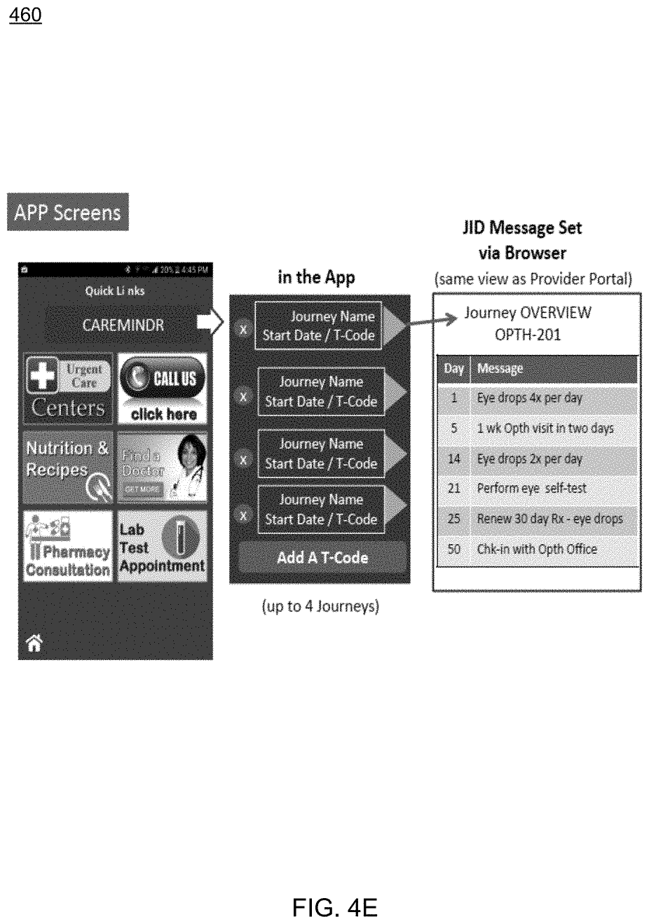

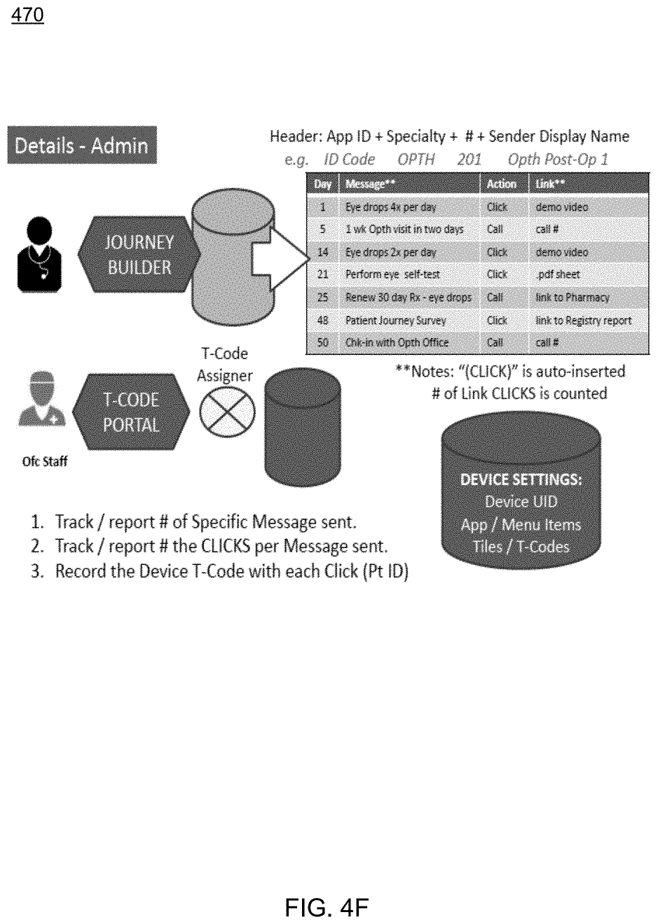

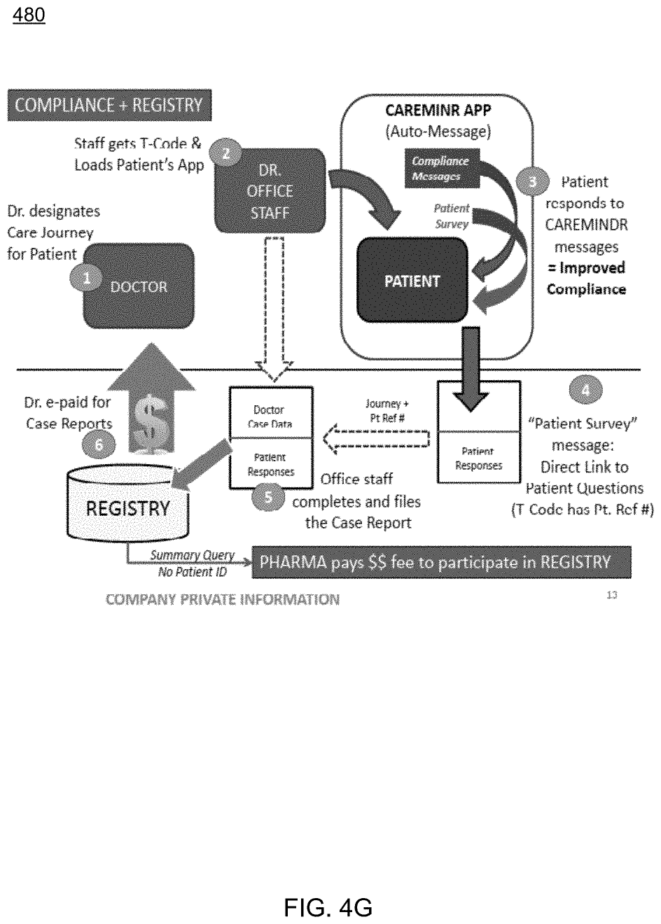

FIG. 4A illustrates an example setup configuration for a new course of treatment according to example embodiments. Referring to FIG. 4A, the illustration 400 includes the basic setup functions of linking a particular journey T-code (unique code) to the message and/or URL to link the application of the user to a customized template, such as a response form, questionnaire, etc. The unique T-code, date, time, response, and other records for each instance may be stored in a patient record managed by the application system database.

FIG. 4B illustrates an example database entry for the new course of treatment according to example embodiments. Referring to FIG. 4B, the example configuration 430 includes a database entry of messages which are organized by a category, in this case ophthalmology, and with a message content, including a link to a response page. The context and add-ons of a particular message may be customized based on a preferred layout or a default layout.

FIG. 4C illustrates a flow diagram 440 configuration for the new course of treatment according to example embodiments. Referring to FIG. 4C, the flow diagram includes a daily batch of messages which are setup to be delivered to one or more assigned patients. The process begins with a trigger to send a message, such as a matured date or time. The process then continues to deliver additional messages once confirmation of delivery is made. If the message is delayed or the response required is not received, the message may be resent as a late message requiring immediate attention. The process may continue to cycle to identify whether any messages are outstanding or have not been confirmed.

FIG. 4D illustrates an example list of messages for the ongoing course of treatment according to example embodiments. Referring to FIG. 4D, in this illustration 450, the various messages intended for a particular patient are illustrated by date. FIG. 4E illustrates an example setup configuration for various courses of treatment according to example embodiments. Referring to FIG. 4E, the configuration 460 includes a menu of options along with a set of potential journeys the user may be assigned to manage the ongoing health care treatment plans for that user. The overview of treatment options and dates are included for reference purposes.

FIG. 4F illustrates an example set of details of an ongoing course of treatment according to example embodiments. Referring to FIG. 4F, the details of the administrator are shown to include a journey builder function based on certain parameters, such as an identification code, specialty, a number and a sender name. The number of messages, responses and actions are recorded to demonstrate the user's interaction with the application and the specific treatment plan(s).

FIG. 4G illustrates an example network configuration of the various third parties involved in the application operation and compliance according to example embodiments. Referring to FIG. 4G, the large-scale network of communications among the integrated platform 480 demonstrates the process initiating with the doctor's office establishing a journey for the patient and assigning a T-code (unique code). The patient's responses are identified along with links and references to third party message links and other information sources.

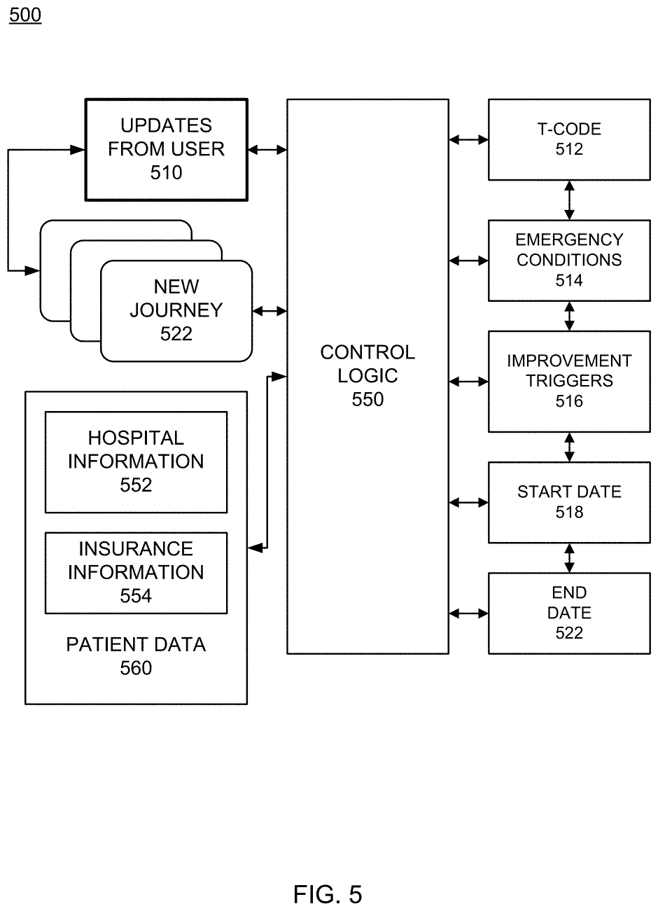

FIG. 5 illustrates a logic module configured to process the input and output parameters of the application according to example embodiments. Referring to FIG. 5, the control logic platform 500 includes a control logic unit 555, such as a processor or other processing entity that may receive updates from a user 510, new journey information 522 and/or patient data 560 including hospital 552, insurance 554, and other information. The logic may be configured to identify and link the unique T-code 512, emergency conditions 514, improvement triggers 516 for optimal changes to the treatment plan, along with dates 518 and new journey information 522 to perform the treatment plan.

In addition, while the term “message” has been used in the description of embodiments of the present application, the application may be applied to many types of network data, such as, packet, frame, datagram, etc. For purposes of this application, the term “message” also includes packet, frame, datagram, and any equivalents thereof. Furthermore, while certain types of messages and signaling are depicted in exemplary embodiments of the application, the application is not limited to a certain type of message, and the application is not limited to a certain type of signaling.

According to example embodiments, a user device, such as a smartphone, cellular phone, tablet device, laptop or other computing device with a memory and processor, may communicate with another computing device and/or a server to provide an integrated communication platform.

Example embodiments provide a computer system programmed to use automated messaging from medical offices to specific patients. The application is not limited to medical procedures and functions and may be used with other configurations for various purposes and services benefiting the end user. Example embodiments include three main computer systems, which work together in an integrated manner including a management platform that controls set-up, functionality, activity reporting, and messaging credentials for the users. An administrative platform which the doctor and doctor's office can access via the internet, and a mobile application that a patient can download into a mobile computer device such as a smartphone or tablet. The management platform acts as the nexus of the system sending outgoing messages on behalf of the healthcare provider and forwarding patient responses to the healthcare provider's administrative platform. The medical office may have a specific identification that is stored within the management platform.

The integrated platform provides a way of checking-in with a patient at prescribed intervals during times between office visits and when undergoing certain treatment that the doctor is providing or overseeing for the patient. The patient dialog may gather relevant information about the status of the patient's conditions or recovery and can be modified or tailored to specifically meet the dialog requirements of the treating physician. Once initiated by the doctor's office, the application operates in an autonomous manner by delivering messages to the patient to prompt responses if needed. The application functions are monitored to assure that the patient replies to the information requests from the doctor, otherwise a no-response alert is sent to the doctor's office. The interactions are recorded and time-stamped, providing an auditable record of the dialog, suitable for insurance billing purposes. The application can also support biometric information from devices that measure certain body functions, such as diabetes glucometers, or blood pressure cuffs, or any sensory readable health care metric. The application may also create a longitudinal record of information for the patient to illustrate week-to-week trends.

Response Alert Tags

As has been stated earlier, this method and system is utilized when a patient visits a healthcare provider for an illness/condition which is diagnosed and treated. The treatment occurs over a period of time and is referred to as a journey. The system tracks a patient's progress along the journey for that illness or condition and solicits health information from the patient at clinically-relevant intervals, across an extended time period to enable evidence based medicine. The specific information sought, the intervals, and the time period duration apply to specific conditions or illnesses for which a specific patient is being treated.

This solicitation for patient information may take the form of queries sent to the patient for information, when the responses to those queries are delivered to the patient's healthcare provider (e.g. physician). The patient's journey may have a number of waypoints occurring at the clinically-relevant intervals. The responses to the queries at these waypoints are meant to determine a patient's progress and status and to present to the healthcare provider evidence upon which to conduct evidence-based medicine. The responses are collected by the system and measured against historical norms for the patient and/or expected answers for similar patients on similar journeys.

In the event of an unexpected response to a query at that waypoint, the response is treated as notable. Notable events may be considered non-urgent or may be considered urgent or emergent. This divergence from the expected response outcome is graded for severity or urgency. If the severity or urgency of the response exceeds a predetermined threshold for that patient for that journey for that illness or condition at that waypoint, an urgent tag is created and sent to the healthcare provider. The grading may be one of an immediate medical action advisory, a follow-up advisory and a medical history review advisory

The information requested in the query is sent in a structured format to allow ease in answering and the response data is delivered to the healthcare provider in a structured data format to facilitate ease in analysis and trend detection.

The response alert tag is a feature that “tags” certain responses provided by the patient as information that requires follow-up or special notice by the patient's healthcare provider. The tag may indicate a level of severity or urgency, thus alerting the provider to information that may need immediate medical action, additional follow-up with the patient or a specific review of the patient's medical history.

The tag may be communicated to the provider through multiple channels depending upon circumstance and urgency and in an immediate manner or in a weekly aggregated format depending in part upon urgency.

Work flow instructions may be electronically linked to a tag, so that the specific healthcare provider that reviews the data will have guidance about the actions to be taken when a tag appears and any escalation of clinical review that might be appropriate.

Each patient for each illness or condition is interacted with by the system at intervals which are relevant to that illness or condition and the queries are sent to determine the patient's progress or status. The received response to the query is measured against an expected response, and anomalies or offsets are noted. If these response anomalies or offsets are larger than a predetermined amount, an urgent or severe issue may need to be addressed. Thus the response is tagged as urgent and may be sent utilizing a priority delivery schedule, a priority delivery indicia on the response and may be made to a priority delivery list determined by the healthcare provider. The response may be tagged as non-urgent if the determined urgency level does not meet the predetermined urgency threshold of the patient for the health related issue.

The structured format allows an overlap of queries so that the patient is not answering multiple identical queries at any one point in time. Additionally, the structured format allows the data to be collected and logged in a structured format and assembled for future review both by the practitioner and the patient to determine trends.

In one example a method, includes requesting via a cloud based system from a patient response to a query and receiving the response to the health related query, determining an urgency level of the response based on the patient health related issue and tagging the response as urgent if the determined urgency level exceeds a predetermined urgency threshold of the patient for the health related issue.

The method also includes providing the urgent tagged response to the health provider, where the urgent tagged response may be sent utilizing a priority delivery schedule, a priority delivery indicia for the response and may be made to a priority delivery list.

The method may also include tagging the response as non-urgent if the determined urgency level does not meet the predetermined urgency threshold of the patient for the health related issue.

If the determined urgency level of the response is such that it rises to the level of a medical emergency, then the primary care physician may be immediately notified as well as emergency services such as 911 and if deemed appropriate, dispatched to the location identified either by the patient or gathered from a location indicator in his mobile device. If the response is deemed critical, in situations where the primary physician is not immediately available, an emergency medical specialist may be placed in active direct communication with the patient. The system would make available to the first responder the query and response to provide context for the escalation.

The response may be graded as to the tagged urgency level of the response, where the grading is at least one of an immediate medical action advisory, a follow-up advisory and a medical history review advisory. A follow-on query may be sent based on the urgent tagged response to give the provider context to the urgent tagged response. As an example, if the patient responds that they have been to the emergency room (ER) that may trigger another set of queries about the ER visit to add context to the response. This second set of queries may determine whether the ER visit was related to conditions or illnesses related to the journey, or whether visit was for a condition unrelated to the journey, but still of interest to the healthcare provider.

In another example a cloud based system links user equipment and a healthcare provider server. The cloud based system requests a response to a query from a patient pertaining to a health related issue, receives the response to the query and determines an urgency level of the response based on the patient health related issue. The system also tags the response as urgent if the determined urgency level exceeds a predetermined urgency threshold of the patient for the health related issue and provides the urgent tagged response to health provider.

The cloud based system may receive via the user equipment a sensor signal provided by a medical device in response to the query. The medical device may be a blood pressure monitor, a glucometer, a pulse meter, a continuous positive airway pressure device, a heart monitor, an implanted medical device and the like.

The cloud based system may receive via the user equipment an audio or text message indicating a medical distress condition in response to the query or may overhear the patient indicating a medical distress condition in conversations or texts in an unsolicited message.

The system may also interpret patient actions in regards to patient historical norms, such as, if the patient is overheard slurring his speech, he may be having a stroke, or if he is discussing that he has pressure in his chest or his left arm is numb, he may be having a heart attack. At this point the system may connect him directly to a medical specialist and take other appropriate action, such as determining his location and dispatching emergency services.

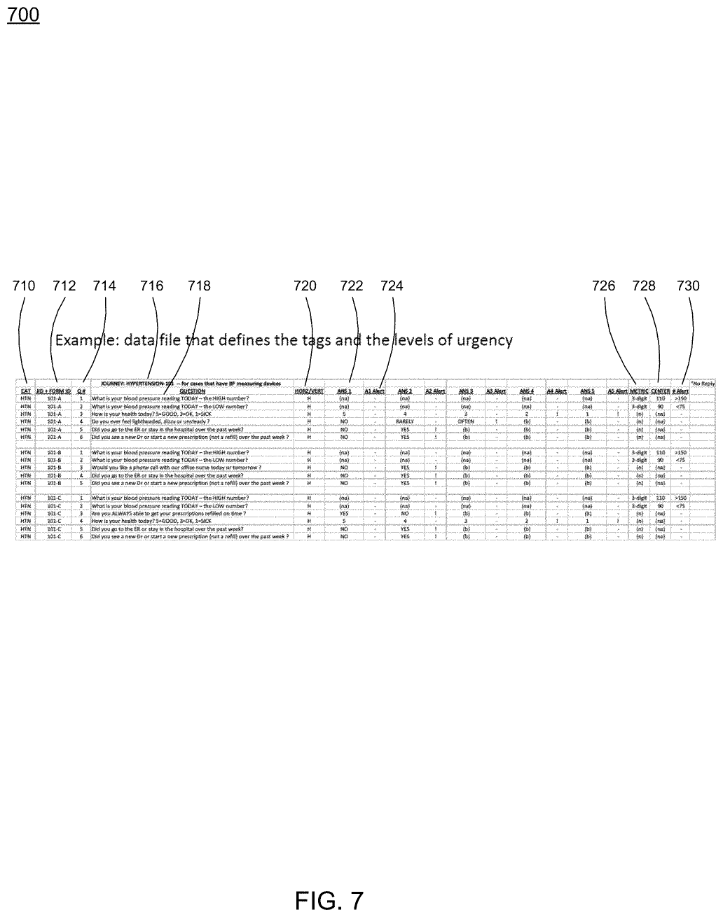

FIG. 7 depicts an example data file 700 that defines the tags and levels of urgency. The parts of the data file include a category of question 710, the journey ID and form ID 712, a question number 714, a patient journey for that specific illness or condition 716 and questions associated with that journey 718. The data layouts may be shown horizontally or vertically 720 which allows viewing of individual answers to queries. The historical and current query response 722 and the determined alert 724 are shown, which in a historical context allows detection of trends. The data output of the metric 726 may be numerical, yes or no, and the like, a median query answer 728 for that patient or that condition and an alert threshold 730 which may be modified by the healthcare profession are shown.

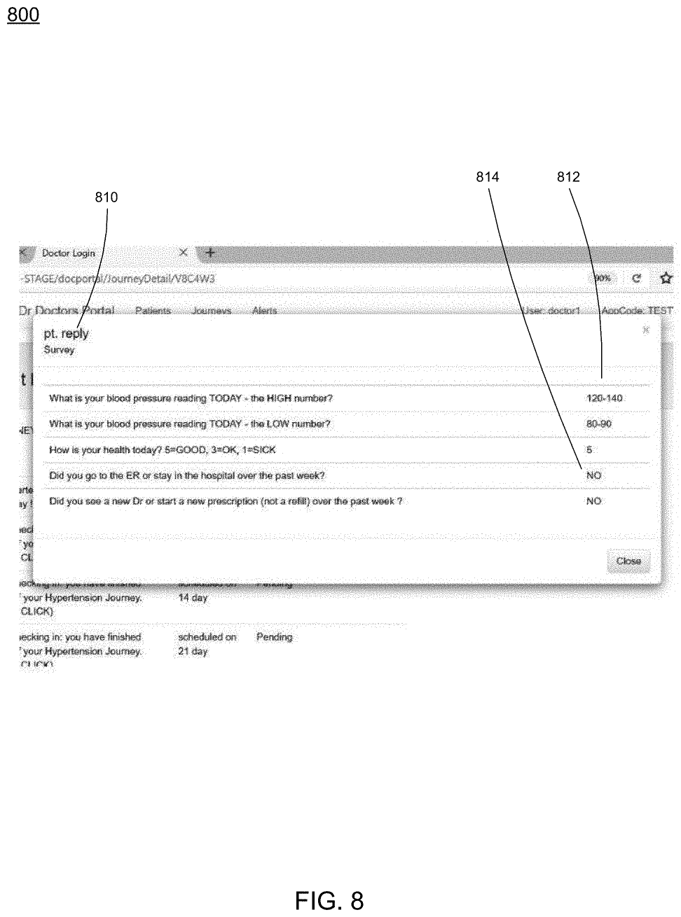

FIG. 8 depicts a patient survey reply 810 having a band of expected replies for blood pressure 812, how the patient feels and whether he went to the emergency room, hospital 814 or has started a new prescription.

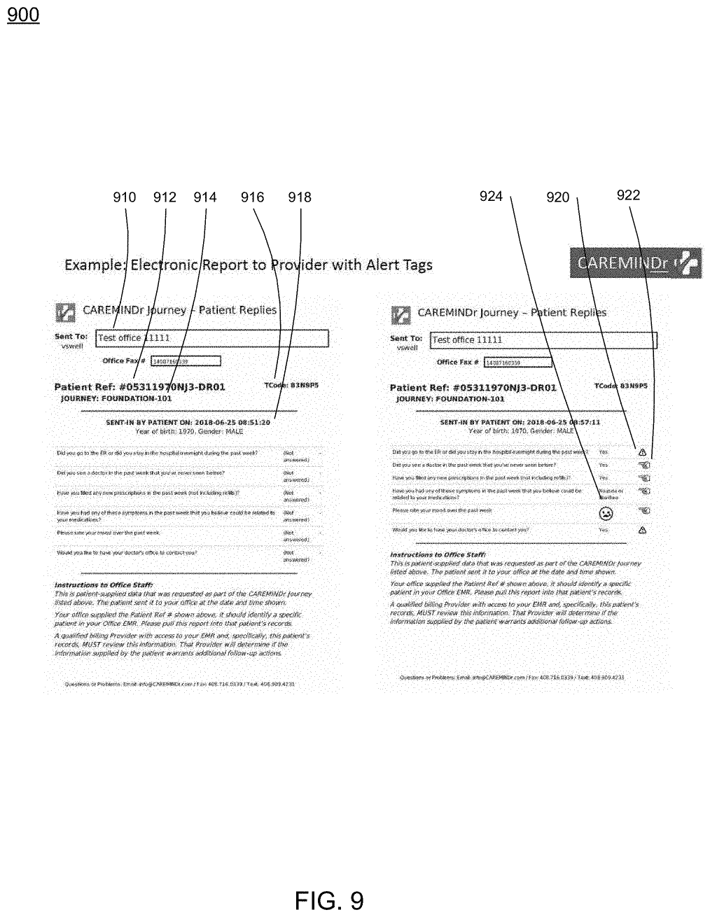

FIG. 9 depicts both a non-responsive and responsive reply set 900. The recipient 910, patient reference number 912, journey ID 914, unique T-Code 916 and timestamp 918 are depicted in the reply. A responsive report having alerts indicates an urgent issue 920, a follow up item 922 and an emoticon 924 indicating a patient feeling is shown.

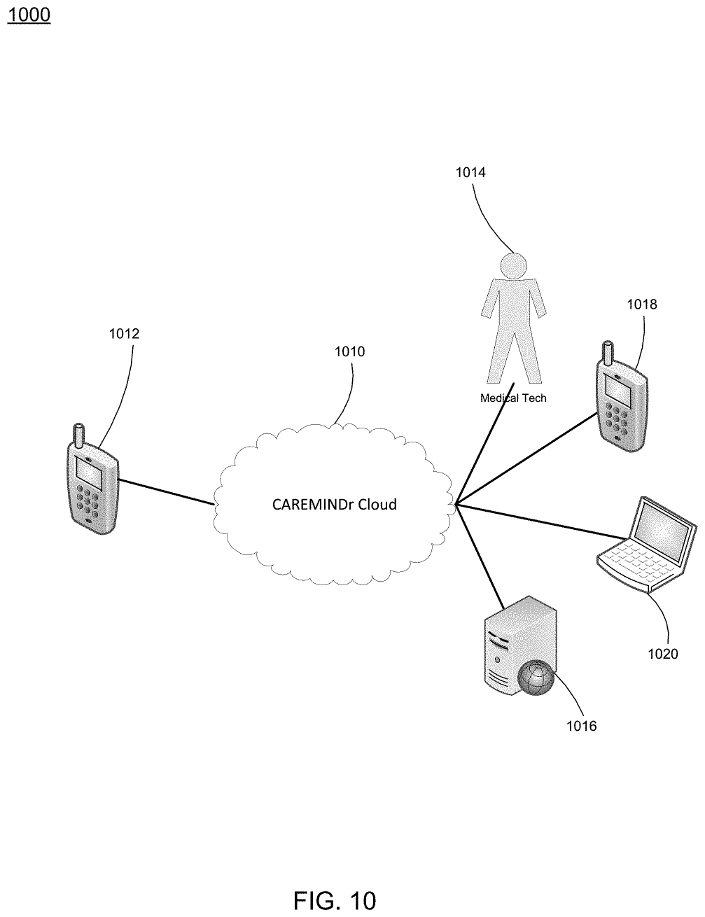

FIG. 10 depicts an example 1000 of communication associated with the alert tag. The cloud based system 1010 sends a request with queries to the patient's communication device 1012 which the patient fills out and returns. The cloud based system 1010 reviews the response and determines whether there are urgent or emergency issues and sends an urgent tagged response to the healthcare provider.

If there is an emergency issue the cloud based system may contact or place the patient in contact with a medical technician 1014 in addition to notifying the healthcare provider by means of the healthcare provider's server 1016, the cloud based system may issue a text or message to the healthcare provider. The communication route from the healthcare provider may be by means of mobile device 1018, computer 1020 or the like. The cloud based system may directly connect the patient via to the patient's communication device 1012 to the healthcare provider under appropriate circumstances. Non-urgent issues are sent to the healthcare provider for later review.

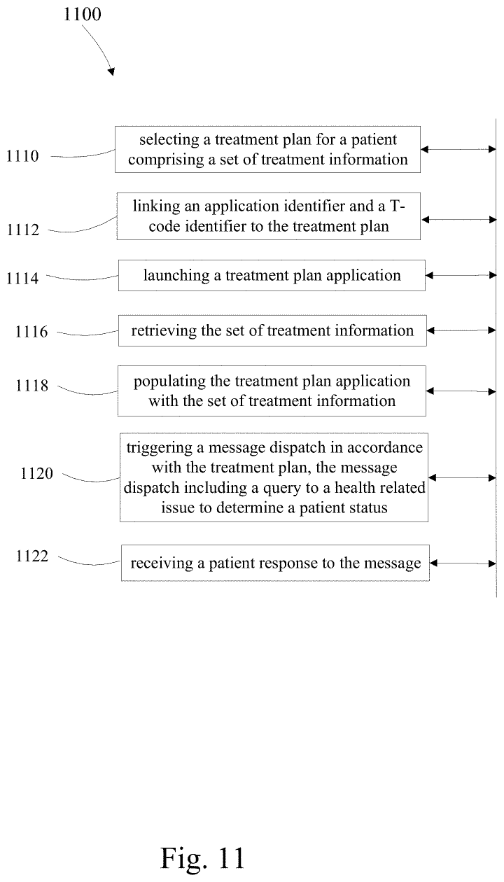

A first example method shown in FIG. 11, 1100, may include, selecting 1110 a treatment plan for a patient comprising a set of treatment information, linking 1112 an application identifier and a T-code identifier to the treatment plan and launching 1114 a treatment plan application. The method further includes retrieving 1116 the set of treatment information, populating 1118 the treatment plan application with the set of treatment information and triggering 1120 a message dispatch in accordance with the treatment plan. The message dispatch includes a query to a health related issue to determine a patient status and the system receives 1122 a patient response to the message.

With respect to the timing of patient responses, the first example method may also include, awaiting the patient response to the message for a late response duration and categorizing the patient response if the patient response is received within the late response duration. If the patient response is not received within the late response duration the method further comprises sending a duplicate message and flagging the patient response as non-responsive if the patient response to the duplicate message is not received within a second late response duration.

The timing of the message dispatches associated with the treatment plan is partly governed by a trigger table. The method may include loading the trigger table having a set of trigger dates based on the treatment plan where the message dispatch is sent according to the set of trigger dates. The method may further include receiving a message start date and receiving an initialization message from a patient mobile device to initiate the treatment plan and to initialize the set of trigger dates in the trigger table.

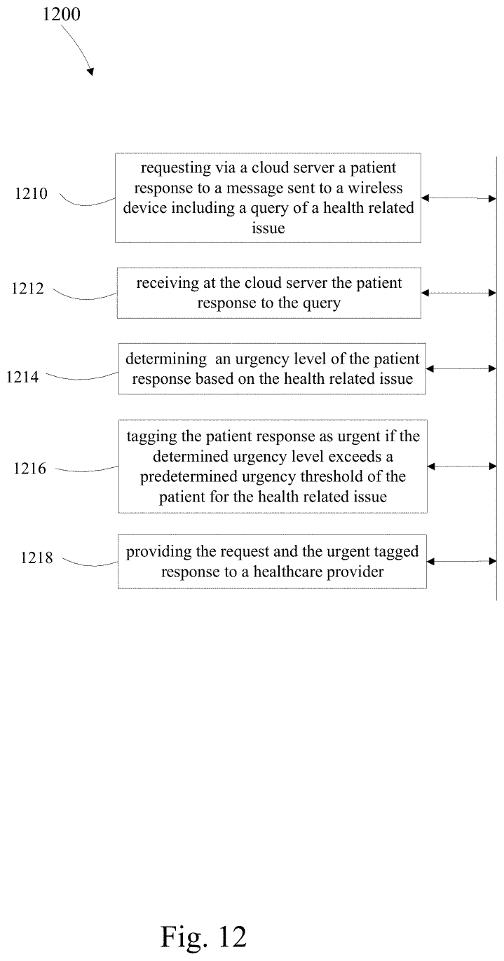

A second example method is shown in FIG. 12 related to response tagging, may include requesting 1210 a patient response to a message including a query of a health related issue and receiving 1212 the patient response to the query. The method then provides determining 1214 an urgency level of the patient response based on the health related issue, tagging 1216 the patient response as urgent if the determined urgency level exceeds a predetermined urgency threshold of the patient for the health related issue and providing 1218 the request and the urgent tagged response to a healthcare provider. The method may also include proposing a set of work flow instructions linked to an urgent or non-urgent tagged response and presenting a clinical escalation review advisory if the response is tagged as urgent.

A first example non-transitory computer readable medium comprising instructions associated with the tagging of responses that, when read by a processor, cause the processor to perform; linking a user equipment and a health care provider server, requesting from a patient pertaining to a health related issue a response to a query and receiving the response to the query. The processor then determines an urgency level of the response based on the patient health related issue, tags the response as urgent if the determined urgency level exceeds a predetermined urgency threshold of the patient for the health related issue and provides the request and the urgent tagged response a healthcare provider.

Journey Log Overview

Evidence based medicine at its heart is patient data centric. The metrics themselves are derived from patient responses. The impact those metrics have on the patient's ownership of his actions is directly related to his recognition of the fact that his actions impact outcomes. Convincing a patient of how his actions impact his health are vital and are one of the core aspects of the journey log, called J-Log.

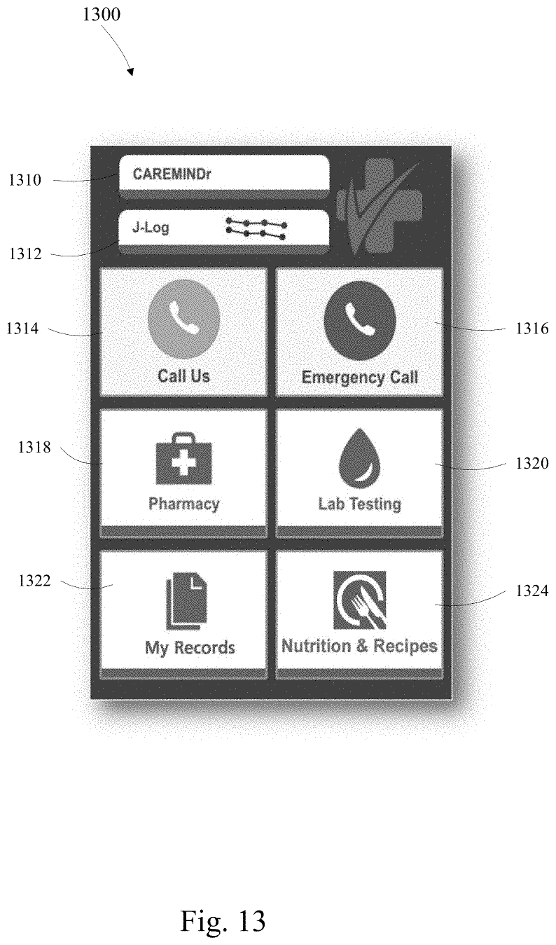

FIG. 13 depicts an example screenshot of a journey log (J-Log) app. The J-Log app has a menu 1310 at the top of the screen and has a journey log data display button 1312. The application includes a button labeled call us 1314 to call the healthcare provider, an emergency call button 1316 to call emergency medical services in case of a patient recognized emergency. The app also has a button 1318 to contact the patient's pharmacy and a lab testing button 1320 to contact their labs in order to schedule or receive data from testing. The app includes a records button 1322 to review their personal medical records and nutrition and recipes button 1324 for healthy nutritional information.

Journey Log Types of Data

The types of data the journey log will utilize will be objective, such as blood pressure and blood glucose levels and subjective, such as, how do you feel, did you sleep well last night, etc. J-Log fuses subjective and objective components so that interactions between actions and not just objective data, but subjective criteria may be established. For example, objective data such as prescription compliance and blood glucose levels may be charted against how the patient states they feel, or whether their sleep habits were affected. In the J-Log the only data reviewed will be that which is input by the patient, i.e. patient supplied data will be the only source of information which is charted. Long term sleep patterns may emerge based on the data from multiple snapshots that have been reviewed by an analysis algorithm.

Journey Log Sources of Data

The data collected, both objective and subjective will be from the patient himself. The data is input based on the questions received from the system. At present the data input is manually entered into a mobile device. It is envisioned that automatic data downloads from biometric devices by means of Bluetooth and the like will be directly downloaded to the system.

Journey Log Analysis of Data

The patient data is closely monitored by the system to give trend alerts if potentially dangerous trends develop, to correct small issues before they become large problems. The trends may be established over days, weeks or months. Additionally, the medical staff will be able to review patient to patient comparisons to determine whether the patient is responding outside of established norms. Artificial intelligence and machine learning will be applied to the inputs and outputs of the data.

Journey Log Presentation of Data to Patient

An interesting ability that almost all humans have is the ability to spot trends. J-Log is a tool which visually indicates the long term trends that the responses indicate. Patients will receive timelines of blood pressure, blood glucose levels and the like. Doctors will be shown multi-input overlays such as prescription compliance and blood pressure or blood glucose. The data may be shared by the patient with family and caregivers, which further reinforces compliance with prescriptions. It is envisioned that patient engagement in viewing their health will encourage compliance and the observation of long term trends are vitally important to medical personnel.

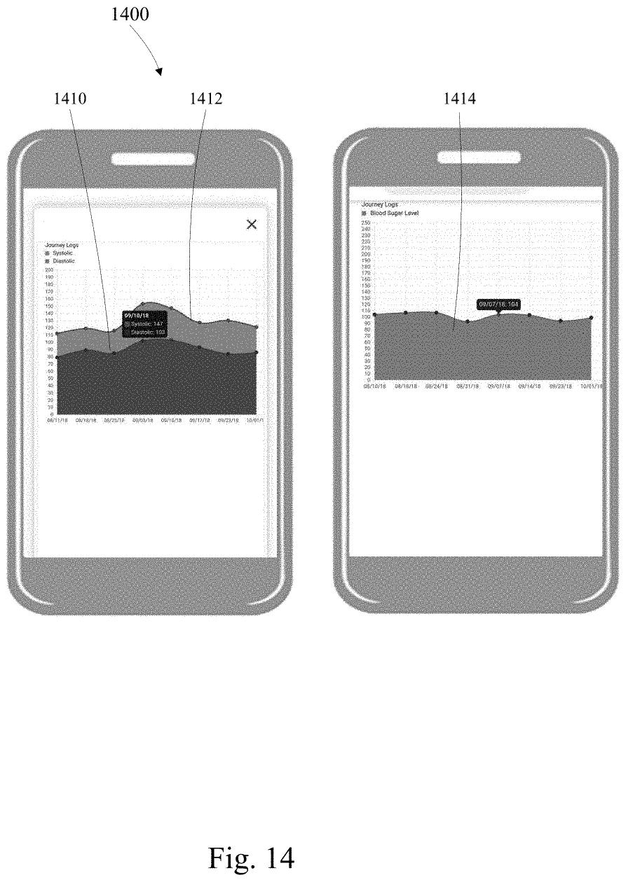

FIG. 14 depicts the presentation of objective data to the patient in the J-Log app, systolic 1412 and diastolic 1410 blood pressure are shown versus date and blood glucose levels 1414 are shown laterally versus date. The data versus time graphs indicates to the patient their historical biometric data trends, to show whether their biometrics are stable or changing over time for the better or worse.

Journey Log Presentation of Data to Health Care Professionals

Doctors and medical staff will receive not only timeline but additionally XY plots showing multi-factor correlations. Multiple outputs may be combined into a single graph to give medical personnel further insight. In J-Log longitudinal components allow snapshots and long term graphs of the charted data and flagged comments. It allows warning signs to be more readily recognized and dealt with and allows reoccurrence of events to be reviewed. The structured database allows presentation to the doctor of both snapshots in time and trending data to show whether the patient is getting better, worse or is about the same. It encourages the patient to stay on the program and encourages behavioral changes. The data allows review of readily understood trends versus single encounter clinical assessments, i.e. it puts the doctor into the virtual life of the patient.

The analysis of the patient provided data allows the system to not only display the individual metrics in time, but allow time offsets between drug compliance and either objective or subjective criteria. As an example, if a patient forgets to take their diabetes medication and two days later suffers a spike in blood sugar for a relatively small carb overload, it may be possible to correlate the increased sensitivity to the previously missed medication. Or, the patient may experience poor sleep or restlessness if diabetes medication is missed two days ago and blood pressure medication was forgotten on the evening before. Additionally, the cross correlation of exercise or missed medication on how the patient feels may be observed by the system. Although people respond in the main to specific criteria, certain patients may be more sensitive to specific criteria that the system would observer and point out to the doctor.

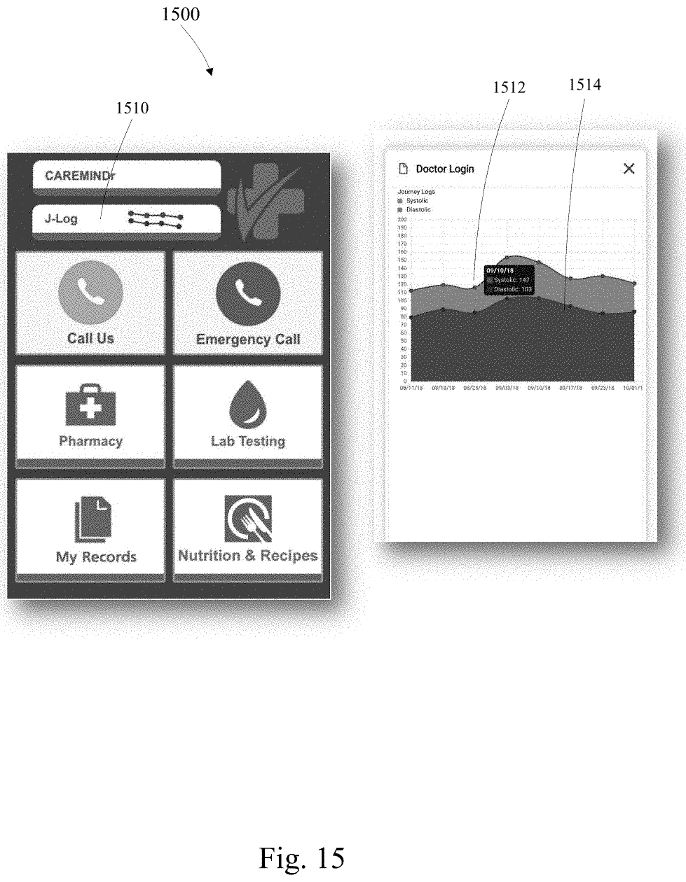

FIG. 15 depicts a doctors' presentation of objective data from the J-Log app. The J-Log app button is shown as 1510 and the data output for blood pressure are shown as 1512 and 1514. The data shown in this graph is a time based display of blood pressure indicating a spike and subsequent return to normal levels.

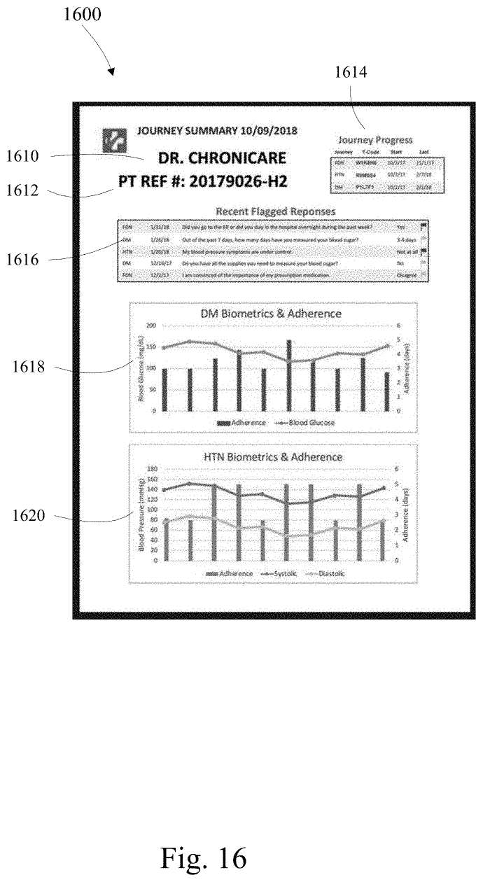

FIG. 16 also depicts a doctors' presentation of objective data from the J-Log app. In this report the medical professional's name is shown as 1610, the patient identifier is 1612, the patient's progress is shown as 1614 and flagged responses are shown in a table 1616. The flagged responses are those which are highlighted for further review by the medical care staff. The patient drug protocol adherence and subsequent blood glucose levels are shown in 1618 and the patient drug protocol and subsequent blood pressure levels are shown in 1620.

Journey Log Alerts for Concerning Trends

Data collection via a mobile device is also increasing, such as a phone's ability to detect heartrate or respiration rates and the ability to determine criteria merely from speaking into the phone. The patient may speak into the phone, measurements sent to the cloud and analyzed and results returned for their review.

Journey Log Virtual Visits

Patient Generated Health Data (PGHD) forms the pool of data from which the health care provider pulls to begin interaction with the patient. The biometrics measured, are measured by the patient. The inclusion of subjective information by the patient, such as how they feel, how they have been sleeping, gives the health care provider further insight and provides part of a context for the objective data. Additional information such as a telephone call between the patient and healthcare provider raise the level of the interaction to a virtual encounter. The healthcare provider may ask for any additional information from the patient, which the patient may respond, “I'm having trouble making decisions”, “food doesn't taste right”, or “I cannot play chords on my guitar” may initiate an entire line of enquiry that would not have been broached otherwise.

If the patient has outside care, for falling and needing stitches, the objective data may provide the data necessary to provide a continuous care transition. The healthcare provider asking why the patient fell down may indicate dizziness, which may indicate a worsening of the base condition that the patient is being treated for. J-Log allows the melding of objective and subjective biometrics to add context and meaning to interactions between the provider and the patient. The collection and display of the data may provide the background data for a high value virtual encounter in which a doctor, noticing a trend may reach out to the patient to confer. The virtual encounter may be via the patient's mobile device which would allow for highly efficient use of both the patient's and doctor's time.

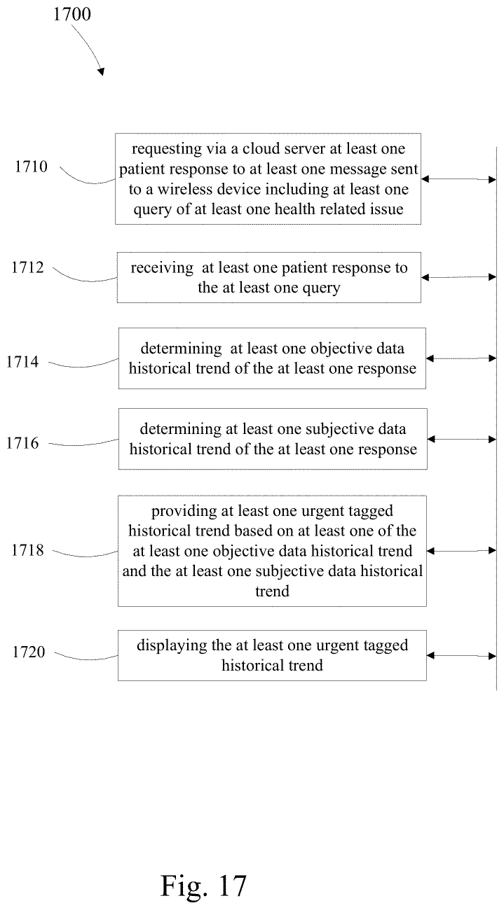

FIG. 17 depicts a first method example of the J-Log system. Example method 1700 includes, requesting 1710 via a cloud server at least one patient response to at least one message sent to a wireless device including at least one query of at least one health related issue, receiving 1712 at least one patient response to the at least one query, determining 1714 at least one objective data historical trend of the at least one response, determining 1716 at least one subjective data historical trend of the at least one response, providing 1718 via the cloud server at least one urgent tagged historical trend based on at least one of the at least one objective data historical trend and the at least one subjective data historical trend, and displaying 1720 the at least one urgent tagged historical trend.

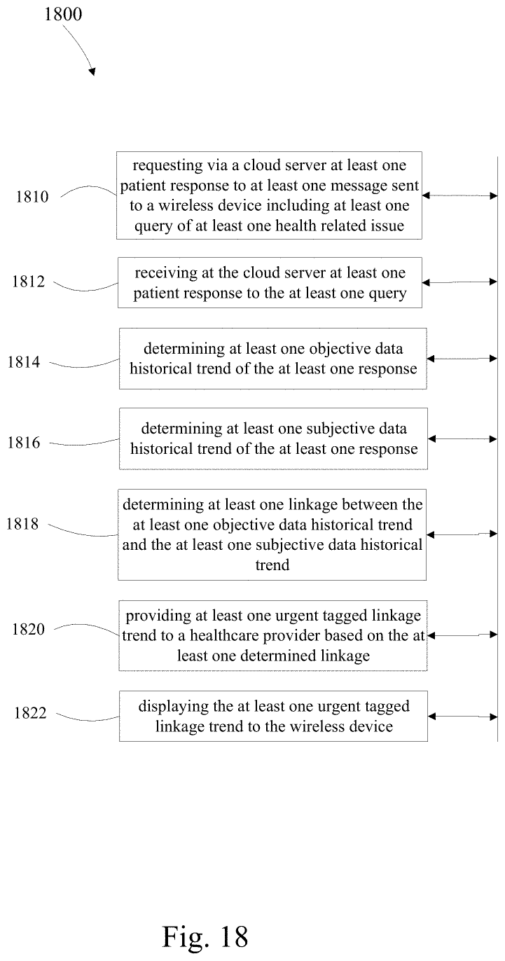

FIG. 18 depicts a second example of the J-Log system. Another example method 1800 includes, requesting 1810 via a cloud server at least one patient response to at least one message at a wireless device including at least one query of at least one health related issue, receiving 1812 at the cloud server at least one patient response to the at least one query, determining 1814 at least one objective data historical trend of the at least one response, determining 1816 at least one subjective data historical trend of the at least one response, determining 1818 at least one linkage between the at least one objective data historical trend and the at least one subjective data historical trend, providing 1820 via the cloud server at least one urgent tagged linkage trend to a healthcare provider based on the at least one determined linkage and displaying 1822 the at least one urgent tagged linkage trend to a wireless device.

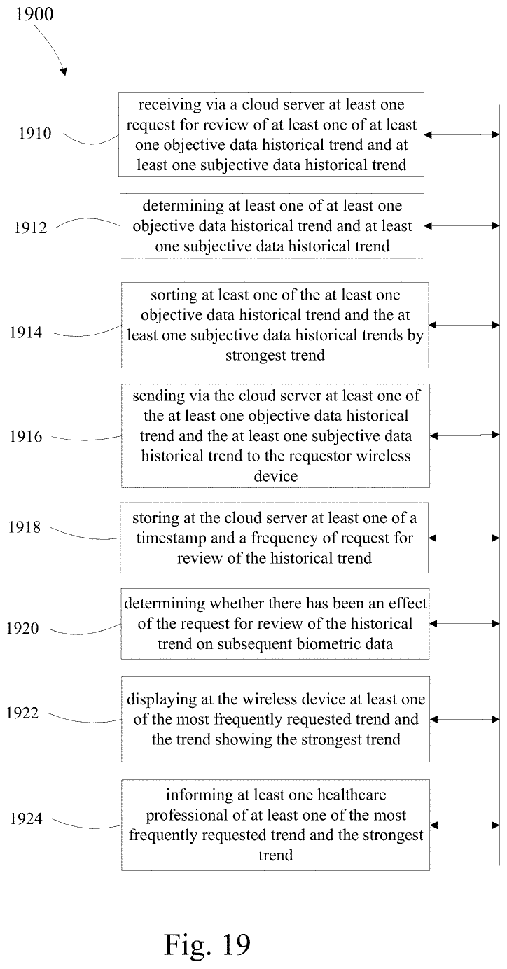

FIG. 19 depicts a third method example of the J-Log system. Example method 1900 includes receiving 1910 at a cloud server at least one request for review of at least one of at least one objective data historical trend and at least one subjective data historical trend, determining 1912 via the cloud server at least one of at least one objective data historical trend and at least one subjective data historical trend and sorting 1914 at least one of the at least one objective data historical trend and the at least one subjective data historical trends by strongest trend. The cloud server performs sending 1916 at least one of the at least one objective data historical trend and the at least one subjective data historical trend to the requestor wireless device and storing 1918 at the cloud server at least one of a timestamp and a frequency of request for review of the historical trend. The cloud server performs determining 1920 whether there has been an effect of the request for review of the historical trend on subsequent biometric data, preferentially displaying 1922 at the wireless device at least one of the most frequently requested trend and the trend showing the strongest trend and informing 1924 at least one healthcare professional of at least one of the most frequently requested trend and the strongest trend.

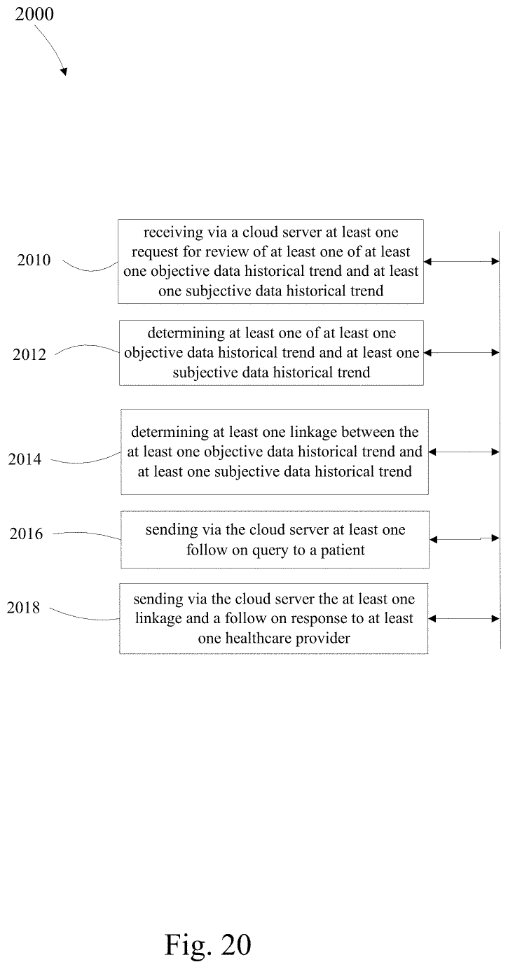

FIG. 20 depicts a fourth method comprising receiving 2010 via a cloud server at least one request for review of at least one of at least one objective data historical trend and at least one subjective data historical trend, determining 2012 at least one of at least one objective data historical trend and at least one subjective data historical trend, determining 2014 at least one linkage between the at least one objective data historical trend and at least one subjective data historical trend, sending 2016 via the cloud server at least one follow on query to a patient and sending 2018 via the cloud server the at least one linkage and a follow on response to at least one healthcare provider.

The operations of a method or algorithm described in connection with the embodiments disclosed herein may be embodied directly in hardware, in a computer program executed by a processor, or in a combination of the two. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

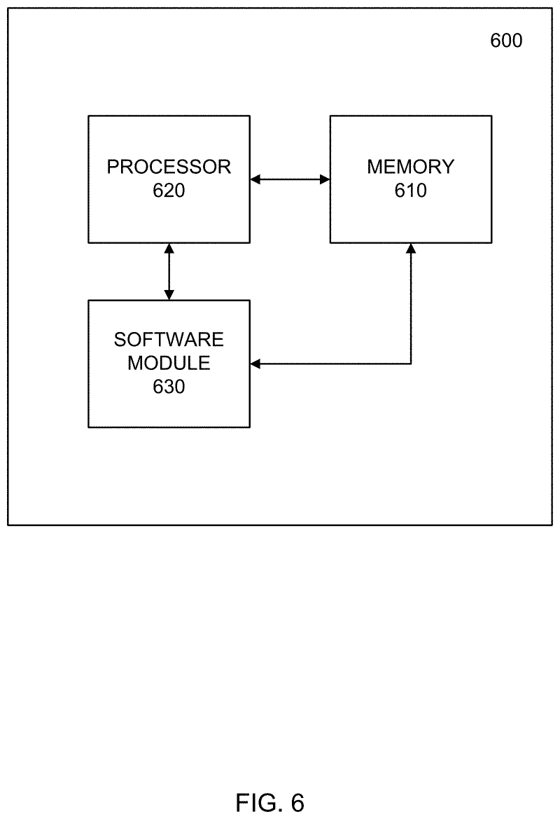

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components. For example, FIG. 6 illustrates an example network element, which may represent any of the above-described network components of the other figures.

As illustrated in FIG. 6, a memory 610 and a processor 620 may be discrete components of the network entity 600 that are used to execute an application or set of operations. The application may be coded in software in a computer language understood by the processor 620, and stored in a computer readable medium, such as, the memory 610. The computer readable medium may be a non-transitory computer readable medium that includes tangible hardware components in addition to software stored in memory. Furthermore, a software module 630 may be another discrete entity that is part of the network entity 600, and which contains software instructions that may be executed by the processor 620. In addition to the above noted components of the network entity 600, the network entity 600 may also have a transmitter and receiver pair configured to receive and transmit communication signals (not shown).

Although an exemplary embodiment of the system, method, and computer readable medium of the present application has been illustrated in the accompanied drawings and described in the foregoing detailed description, it will be understood that the application is not limited to the embodiments disclosed, but is capable of numerous rearrangements, modifications, and substitutions without departing from the spirit or scope of the application as set forth and defined by the following claims. For example, the capabilities of the system of the various figures can be performed by one or more of the modules or components described herein or in a distributed architecture and may include a transmitter, receiver or pair of both. For example, all or part of the functionality performed by the individual modules, may be performed by one or more of these modules. Further, the functionality described herein may be performed at various times and in relation to various events, internal or external to the modules or components. Also, the information sent between various modules can be sent between the modules via at least one of: a data network, the Internet, a voice network, an Internet Protocol network, a wireless device, a wired device and/or via plurality of protocols. Also, the messages sent or received by any of the modules may be sent or received directly and/or via one or more of the other modules.

One skilled in the art will appreciate that a “system” could be embodied as a personal computer, a server, a console, a personal digital assistant (PDA), a cell phone, a tablet computing device, a smartphone or any other suitable computing device, or combination of devices. Presenting the above-described functions as being performed by a “system” is not intended to limit the scope of the present application in any way, but is intended to provide one example of many embodiments of the present application. Indeed, methods, systems and apparatuses disclosed herein may be implemented in localized and distributed forms consistent with computing technology.

It should be noted that some of the system features described in this specification have been presented as modules, in order to more particularly emphasize their implementation independence. For example, a module may be implemented as a hardware circuit comprising custom very large scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together, but may comprise disparate instructions stored in different locations which, when joined logically together, comprise the module and achieve the stated purpose for the module. Further, modules may be stored on a computer-readable medium, which may be, for instance, a hard disk drive, flash device, random access memory (RAM), tape, or any other such medium used to store data.

Indeed, a module of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

It will be readily understood that the components of the application, as generally described and illustrated in the figures herein, may be arranged and designed in a wide variety of different configurations. Thus, the detailed description of the embodiments is not intended to limit the scope of the application as claimed, but is merely representative of selected embodiments of the application.

One having ordinary skill in the art will readily understand that the application as discussed above may be practiced with steps in a different order, and/or with hardware elements in configurations that are different than those which are disclosed. Therefore, although the application has been described based upon these preferred embodiments, it would be apparent to those of skill in the art that certain modifications, variations, and alternative constructions would be apparent, while remaining within the spirit and scope of the application. In order to determine the metes and bounds of the application, therefore, reference should be made to the appended claims.

While preferred embodiments of the present application have been described, it is to be understood that the embodiments described are illustrative only and the scope of the application is to be defined solely by the appended claims when considered with a full range of equivalents and modifications (e.g., protocols, hardware devices, software platforms etc.) thereto.

What is claimed is:

1. A method, comprising:
requesting via a cloud server at least one patient response to at least one message sent to a wireless device including at least one query of at least one health related issue;
receiving at least one patient response to the at least one query;
determining at least one objective data historical trend of the at least one response;
determining at least one subjective data historical trend of the at least one response;
providing via the cloud server at least one urgent tagged historical trend based on at least one of the at least one objective data historical trend and the at least one subjective data historical trend; and
displaying the at least one urgent tagged historical trend.

2. The method of claim 1 further comprising determining at least one linkage between the at least one objective data historical trend and the at least one subjective data historical trend.

3. The method of claim 2 further comprising providing via the cloud server at least one urgent tagged linkage trend to a healthcare provider based on the at least one determined linkage.

4. The method of claim 2, further comprising sending via the cloud server the at least one linkage and a follow on response to at least one healthcare provider.

5. The method of claim 1 further comprising receiving at the cloud server at least one request for review of at least one of at least one objective data historical trend and at least one subjective data historical trend.

6. The method of claim 5 further comprising sorting at least one of the at least one objective data historical trend and the at least one subjective data historical trends by strongest trend.

7. The method of claim 1 further comprising sending at least one of the at least one objective data historical trend and the at least one subjective data historical trend to a requestor wireless device.

8. The method of claim 7 further comprising storing via the cloud server at least one of a timestamp and a frequency of request for review of the historical trend.

9. The method of claim 1 further comprising determining whether there has been an effect of the request for review of the historical trend on subsequent biometric data.

10. The method of claim 1, further comprising displaying at the wireless device at least one of the most frequently requested trend and the trend showing the strongest trend.

11. The method of claim 1, further comprising informing at least one healthcare professional of at least one of the most frequently requested trend and the strongest trend.

12. The method of claim 1, further comprising sending via the cloud server at least one follow on query to a patient.

13. A non-transitory computer readable medium comprising instructions that, when read by a processor, cause the processor to perform:
requesting via a cloud server at least one patient response to at least one message sent to a wireless device including at least one query of at least one health related issue;
receiving at least one patient response to the at least one query;
determining at least one objective data historical trend of the at least one response;
determining at least one subjective data historical trend of the at least one response;
providing via the cloud server at least one urgent tagged historical trend based on at least one of the at least one objective data historical trend and the at least one subjective data historical trend; and
displaying the at least one urgent tagged historical trend.

14. The non-transitory computer readable medium of claim 13, further comprising further comprising sorting at least one of the at least one objective data historical trend and the at least one subjective data historical trends by strongest trend.

15. The non-transitory computer readable medium of claim 13, further comprising:
determining at least one linkage between the at least one objective data historical trend and the at least one subjective data historical trend; and
providing via the cloud server at least one urgent tagged linkage trend to a healthcare provider based on the at least one determined linkage.

16. The non-transitory computer readable medium of claim 13, further comprising determining whether there has been an effect of the request for review of the historical trend on subsequent biometric data.

17. The non-transitory computer readable medium of claim 13, further comprising:
displaying at the wireless device at least one of the most frequently requested trend and the trend showing the strongest trend; and
informing at least one healthcare professional of at least one of the most frequently requested trend and the strongest trend.

18. A system, comprising:
at least one cloud based processor; and
at least one memory electrically coupled to the at least one processor and storing an application,
wherein the processor performs operations to:
request via a cloud server at least one patient response to at least one message sent to a wireless device including at least one query of at least one health related issue;
receive at least one patient response to the at least one query;
determine at least one objective data historical trend of the at least one response;
determine at least one subjective data historical trend of the at least one response;
provide via the cloud server at least one urgent tagged historical trend based on at least one of the at least one objective data historical trend and the at least one subjective data historical trend; and
display the at least one urgent tagged historical trend.

19. The system of claim 18, wherein the processor further performs an operation to:
determine at least one linkage between the at least one objective data historical trend and the at least one subjective data historical trend; and
provide via the cloud server at least one urgent tagged linkage trend to a healthcare provider based on the at least one determined linkage.

20. The system of claim 18, wherein the processor further performs an operation to:
display at the wireless device at least one of the most frequently requested trend and the trend showing the strongest trend; and
inform at least one healthcare professional of at least one of the most frequently requested trend and the strongest trend.
